(19) United States
(12) Patent Application Publication
(10) Pub. No.: US 2011/0085950 A1
(43) Pub. Date: Apr. 14, 2011

(54) CENTRIFUGAL FORCE BASED MICROFLUIDIC SYSTEM AND BIO CARTRIDGE FOR THE MICROFLUIDIC SYSTEM

(75) Inventors: Beom Seok LEE, Hwaseong-si (KR); Jeong Gun LEE, Seoul (KR); Yoon Kyoung CHO, Suwon-si (KR); Ki Eun KIM, Seoul (KR); Jung Nam LEE, Incheon (KR)

(73) Assignee: SAMSUNG ELECTRONICS CO., LTD., Suwon-si (KR)

(21) Appl. No.: 12/575,635

(22) Filed: Oct. 8, 2009

(51) Int. Cl. G01N 33/50; B01L 3/00; G01N 1/38; G01N 21/77

(52) U.S. Cl. 422/504; 422/72; 422/537; 422/540

(57) ABSTRACT

A microfluidic system based on centrifugal force and a bio cartridge for the microfluidic system are provided. The system includes a spindle motor, a rotatable frame detachably mounted on the motor and having a plurality of cells separated by partition walls, and the bio cartridge detachably accommodated in one of the plurality of cells. The bio cartridge includes a chamber for storing a fluid, a channel for transporting the fluid, and a valve for controlling the flow of the fluid. The valve may include a phase transition material, and exothermic minute particles dispersed in the material and generating heat when energy is applied thereto.

### BACKGROUND

1. Field

A microfluidic system based on centrifugal force, which is employed in a field of microfluidics is provided.

2. Description of the Related Art

A microfluidic structure used for a work with a small quantity of fluid in a field of microfluidics may generally include chambers retaining a small quantity of fluid, channels through which the fluid flows, valves controlling the flow of the fluid, and a variety of functional units receiving the fluid and performing predetermined operations. A bio-chip refers to a device configured to perform several tests on a small chip including a biochemical reaction test. Especially, a lab-on-a-chip is a device configured to perform several steps of a process and an operation on one chip.

Making a fluid flow within a microfluidic structure requires an operational pressure, which is usually exerted as capillary pressure or from an additional pump. Recently, microfluidic devices which have a microfluidic structure arranged on a disk-shaped platform and are operated based on centrifugal force have been suggested. These devices microfluidic may be referred to as a lab compact disk (CD) or a lab-on-a-CD.

Such a microfluidic device which operates based on centrifugal force performs a test of a sample reaction depending on a particular application such as immune serum testing and genetic testing. Generally, the microfluidic device includes a plurality of test units for repeatedly performing the same or different tests several times. However, a problem of wasting resources occurs if only some (not all) of test units are used, and then the microfluidic device having the unused test units is discarded. On the other hand, if the microfluidic device in which only some of test units have been used is set aside without being discarded to later utilize the unused test units, the unused test units may become contaminated by the used test units. Even if the used test units do not the unused test units, a residue of the previously used sample in the used units may cause a test performer to be uncomfortable with using the unused test units of the microfluidic device.

Further, a disk-shaped microfluidic device includes a number of layers of substrates adhered thereto by ultrasonic welding or other bonding methods, but the adhesion becomes more difficult to form and more unreliable as the area of the adhesion is larger.

### SUMMARY

One or more exemplary embodiments provide a bio cartridge having a test unit, a microfluidic device and a microfluidic system based on centrifugal force having the bio cartridge.

According to an aspect of one or more exemplary embodiments, there is provided a microfluidic system based on centrifugal force, the system including a spindle motor, a rotatable frame detachably mounted on the motor and having a plurality of cells separated by partition walls, and a bio cartridge detachably accommodated in at least one of the plurality of cells, and a bio cartridge for the microfluidic system. The bio cartridge includes a chamber for storing a fluid, a channel for transporting the fluid, and a valve for controlling the flow of the fluid.

The valve may include a phase transition material, and exothermic minute particles dispersed in the material and generating heat by energy provided from the outside. The system may further include an external energy source for providing energy to the valve so that, by heat generated from an exothermic reaction of the minute particles, the phase transition material undergoes a phase transition to liquidize itself.

The minute particles may be minute metal oxides.

The phase transition material may be wax, gel, or thermoplastic resin.

The energy source may be configured to emit electromagnetic waves to the valve.

The system may further include a dummy cartridge detachably accommodated in at least one of the cells which are not loaded with the bio cartridge, so as to control the rotational balance of the frame.

The cell may be formed in a fanwise shape around the rotational center of the frame, and the bio cartridge may be formed in a fanwise shape corresponding to the shape of the cell.

The frame may include at least one hook member which detachably secures the bio cartridge in the cell.

The system may further include a cover member which detachably secures the bio cartridge to the cell by coupling with the frame and closing the cell.

The bio cartridge may further include a test kit detachably mounted on the bio cartridge and having a test strip determining the existence of a particular substance.

### BRIEF DESCRIPTION OF THE DRAWINGS

The above and other aspects of the disclosed exemplary embodiments will be more apparent from the following detailed description taken in conjunction with the accompanying drawings in which:

FIG. 1 is an exploded perspective view of the microfluidic system according to an exemplary embodiment;

FIG. 2 is a view explaining a usage of the system of FIG. 1;

FIG. 3 is an exploded perspective view of a microfluidic system according to another exemplary embodiment; and

FIG. 4 is an exploded perspective view of a microfluidic system according to another exemplary embodiment.

### DETAILED DESCRIPTION

Exemplary embodiments now will be described more fully hereinafter with reference to the accompanying drawings, in which exemplary embodiments are shown. This disclosure may, however, be embodied in many different forms and should not be construed as limited to the exemplary embodiments set forth therein. Rather, these exemplary embodiments are provided so that this disclosure will be thorough and complete, and will fully convey the scope of this disclosure to those skilled in the art. In the description, details of well-known features and techniques may be omitted to avoid unnecessarily obscuring the presented embodiments.

The terminology used herein is for the purpose of describing particular embodiments only and is not intended to be limiting of this disclosure. As used herein, the singular forms “a”, “an” and “the” are intended to include the plural forms as well, unless the context clearly indicates otherwise. Furthermore, the use of the terms a, an, etc. does not denote a limitation of quantity, but rather denotes the presence of at least one of the referenced item. The use of the terms “first”, “second”, and the like does not imply any particular order, but they are included to identify individual elements. Moreover, the use of the terms first, second, etc. does not denote any order or importance, but rather the terms first, second, etc. are used to distinguish one element from another. It will be further understood that the terms “comprises” and/or “comprising”, or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof.

Unless otherwise defined, all terms (including technical and scientific terms) used herein have the same meaning as commonly understood by one of ordinary skill in the art. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

In the drawings, like reference numerals in the drawings denote like elements. The shape, size and regions, and the like, of the drawing may be exaggerated for clarity.

Hereafter, a microfluidic system based on centrifugal force, and a bio cartridge for the system, are explained in detail according to embodiments.

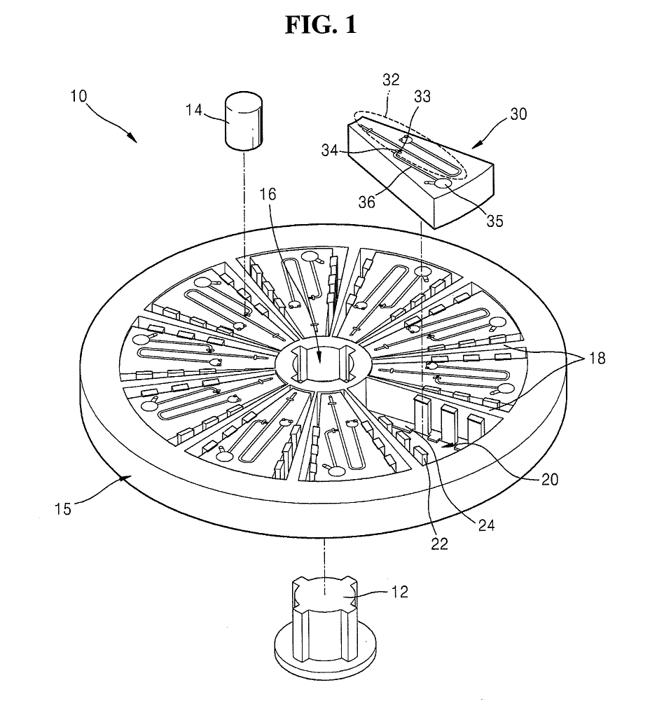

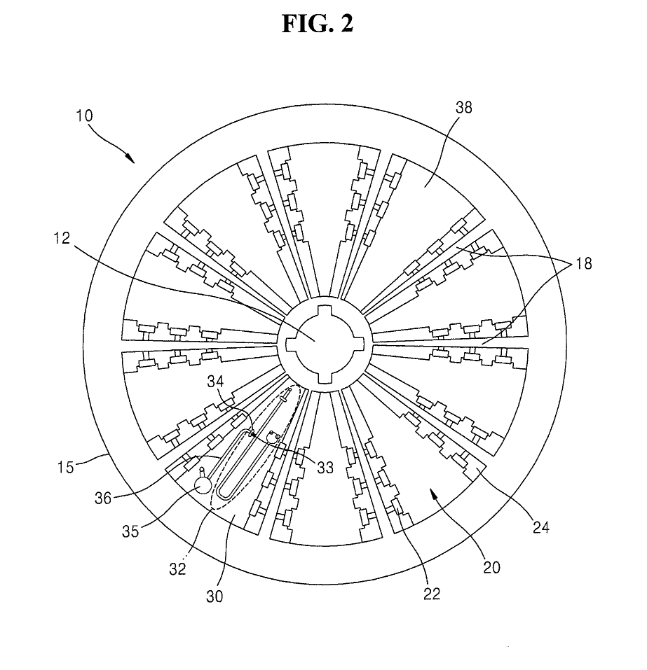

FIG. 1 is an exploded perspective view of the microfluidic system according to an exemplary embodiment, and FIG. 2 is a view explaining a usage of the system of FIG. 1.

As shown in FIG. 1, a microfluidic system 10 according to an exemplary embodiment includes a spindle motor 12, a rotatable frame 15 detachably connected to the motor 12, and at least one bio cartridge 30 detachably mounted in the frame 15.

The frame 15 includes a mounting hole 16 which is provided at the center of the frame 15 and accommodates the spindle motor 12, and a plurality of partition walls 18 extending radially from the center of the frame 15. The frame 15 also includes a plurality of cells 20 defined by and separated by the walls 18. Each of the cells 20 is shaped as a sector or a fan and has the same dimensions. Each of the cells 20 has a fixing portion including hook members 22 detachably fixing the bio cartridge 30 in the cell, and a bracket 24 supporting the bio cartridge 30.

The bio cartridge 30 is mounted in one of the cells 20 of the frame 15. The bio cartridge 30 has a sector or fan shape corresponding to the shape of the cell 20. The bracket 24 supports the bio cartridge 30, and the hook members 22 fix the bio cartridge 30 in the cell 20 and prevent the bio cartridge 30 from becoming unintentionally detached from the cell 20. The bio cartridge 30 may be removed from the cell 20 of the frame 15 by deforming the hook members 22 outwards and lifting up the bio cartridge 30.

The bio cartridge 30 includes a test unit including a chamber storing a small quantity of fluid to be tested, a channel transporting the fluid, and a valve controlling the flow of the fluid. Specifically, as depicted in FIGS. 1 and 2, the bio cartridge 30, which may be utilized for a blood-sugar test by way of example, includes a separation unit 32 centrifugally separating a sample such as whole blood (WB), and a reaction chamber 35 storing a reagent, which will react with a particular material, e.g., glucose contained in serum extracted from the unit 32, thereby determining the existence and the quantity of the particular material. The bio cartridge 30 further includes a channel 36 connecting the unit 32 with the chamber 35, and a valve 33 controlling the flow of the fluid through the channel 36.

The valve 33 opens the channel 36 under a certain condition while it normally closes the channel 36. The valve 33 includes a phase transition material, which remains in a solid phase at normal temperature, and a number of exothermic minute particles dispersed in the phase transition material. The phase transition material may be wax. When heated, the wax melts down and transits into a liquid phase, expanding its volume. The wax may be selected from paraffin wax, microcrystalline wax, synthetic wax, natural wax, etc.

Alternatively, the phase transition material may be gel or thermoplastic resin. The gel may be selected from polyacrylamides, polyacrylates, polymethacrylates, polyvinylamides, etc. The thermoplastic resin may be selected from cyclic olefin copolymer (COC), polymethylmethacrylate (PMMA), polycarbonate (PC), polystyrene (PS), polyoxymethylene (POM), perfluoralkoxy (PFA), polyvinylcholoride (PVC), polypropylene (PP), polyethylene terephthalate (PET), polyetheretherketone (PEEK), polyamide (PA), polysulfone (PSU), polyvinylidene fluoride (PVDF), etc.

The exothermic minute particles range from tens to hundreds of nanometer in diameter so as to pass freely through the channel 36 having a depth of about 0.1 mm, for example. The particles have the exothermic characteristic that their temperatures rise radically due to an energy, which is provided by, for example, emitting a laser beam. The particles may be ferromagnetic minute metal oxide particles such as iron oxide.

The minute particles may be stored in a state of being dispersed evenly in carrier oil. In such a case, in order to be diffused in the carrier oil, the particles may have a molecular structure consisting of a metallic core and a surfactant surrounding the metallic core. A filler for the valve may be prepared by mixing the liquidized phase transition material with the carrier oil in which the minute particles are dispersed. The liquidized filler for the valve is injected and hardened, thereby forming the valve 33 that closes the channel 36.

When energy is provided to the valve 33, e.g., by emitting a laser, the exothermic minute particles generate heat rapidly, and then the phase transition material is rapidly liquidized by the heat. The liquidized filler is discharged to a drain 34 provided on the channel 36, thereby opening the channel 36 so that the fluid flows. The microfluidic system 10 further includes an external energy source 14 for applying energy to the valve 33. The energy source 14 may be configured to emit electromagnetic waves to the valve 33. Specifically, the energy source 14 may include a laser source such as a laser diode to emit a laser to the valve 33.

The bio cartridge 30 may further include a buffer chamber (not shown) for diluting the sample extracted from the separating unit 32 by mixing the sample with a diluent before the sample is transported to the reaction chamber 35. Moreover, the bio cartridge 30 may further have a blank chamber (not shown) filled with distilled water, which functions as a control group against the reaction chamber 35 in which the sample reaction takes place.

The structure and configuration of the bio cartridge, illustrated in the figures herewith, is merely exemplary, and may vary according to the kind of the sample, the use of the bio cartridge, etc.

The bio cartridge 30 may be made with fan-shaped upper and lower substrates (not shown). In other words, after channels, chambers, etc. are formed on either the bottom side of the upper substrate or the top side of the lower substrate, the bio cartridge 30 may be formed by adhering the upper substrate to the lower substrate. Since the bio cartridge 30 is equipped with a single test unit for a blood-sugar test, it has a smaller size than a usual disk-shaped microfluidic device. Thus, the area of the adhesion surface between the substrates becomes smaller than that of a typical disk-shaped microfluidic device, thereby reducing a possibility of faulty adhesion when the substrates are adhered to each other, for example, by ultrasonic welding. The bio cartridge 30 is disposable, and will thus be discarded after it is utilized once for a particular use such as a blood-sugar test.

When a particular test is performed using the microfluidic device 10, the bio cartridges 30 may be mounted not only in all of the cells 20 of the frame 15, as shown in FIG. 1, but also in only some of the cells 20, as shown in FIG. 2. In FIG. 2, only one cartridge 30 is mounted on the frame 15. In this case, rotating the frame 15 with some empty cells 20 may lead to an unreliable test result due to the imbalance of the frame, and also cause a malfunction of the spindle motor 12 or the frame 15. Therefore, in order to control the balance in rotation, a dummy cartridge 38, which has the same shape and weight as the bio cartridge 30, is mounted in the cell 20 on the side opposite to the cell 20 accommodating the bio cartridge 30.

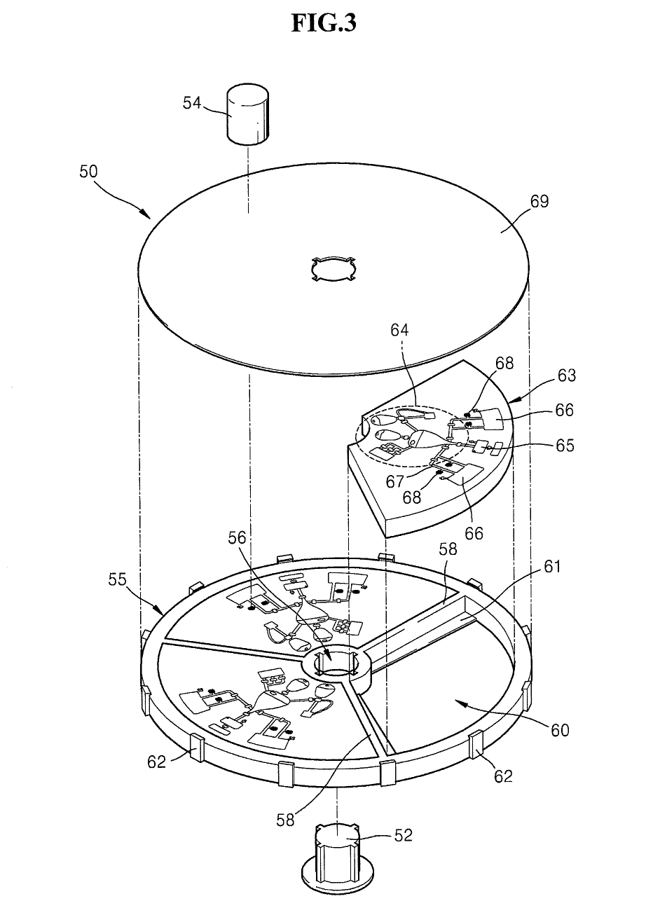

FIG. 3 is an exploded perspective view of a microfluidic system according to another exemplary embodiment.

As shown in FIG. 3, a microfluidic system 50 according to another exemplary embodiment includes a spindle motor 52, a rotatable frame 55 detachably coupled to the motor 52, at least one bio cartridge 63 detachably mounted in the frame 55, and a cover 69 connected to the frame 55.

The frame 55 has a mounting hole 56 at its center, into which the spindle motor 52 is inserted, a plurality of partition walls 58 extending radially from that center, and a plurality of cells 60 separated identically by the walls 58. The cells 60 are formed in fanwise shape, and include a bracket 61 for supporting the bio cartridge 63.

The bio cartridge 63 is mounted in at least one of the cells 60. The bio cartridge 63 has a fanwise shape corresponding to the shape of the cell 60. The bio cartridge 63 is inserted into the cell 60, and then is supported by the bracket 61. The frame 55 includes hook members 62 disposed along its circumference for detachably connecting the cover 69 to the frame 55. When the bio cartridge 63 is mounted in the cell 60 and the cover 69 lies closely onto the upper side of the frame 55, the cover 69 is fixed on the frame 55 by the hook members 62 to close the cells 60, thereby securing the bio cartridges 63 in the cells 60. When the hook members 62 are deformed outwards and the cover 69 is removed from the frame 55, the cells 60 are opened, thereby making it possible to remove cartridges 55 from the frame 55.

In the exemplary embodiment shown in FIG. 3, the hook members 62 arranged around the circumference of the frame 55 are used to secure the cover 69, but this is only exemplary. In another exemplary embodiment, hook members may be disposed at both sides of the frame 55, and the cover 69 may be slid from the side of the frame 55 and fixed between the hook members.

The bio cartridge 63 has a chamber retaining a small quantity of fluid, a channel transporting the fluid, and a valve controlling the flow of the fluid. Specifically, the bio cartridge 63 is a disposable one used for a protein test such as a hepatitis virus test, and will be discarded when used once for a particular purpose. The bio cartridge 63 is provided with a separating unit 64 for separating a particular protein, e.g., a hepatitis virus, from a sample, e.g., whole blood (WB), a reaction chamber 65 storing a substrate that make it possible to distinguish the existence and the amount of that protein, and a waste chamber 66 discharging the remains irrelevant to the reaction. The bio cartridge 63 also includes a channel 67 connecting the separating unit 64 to the waste chamber 66, and a valve 68 controlling the flow of the fluid through the channel 67.

The valve 68 closes the channel 67 under a certain condition. The valve includes a phase transition material, which remains in a solid phase at normal temperature, and a number of exothermic minute particles dispersed in the phase transition material. A valve filler for forming the valve 68 is the same as the filler for the valve 33 in FIG. 1, and thus the a description thereof will be omitted. The valve 68 may be formed by injecting the liquidized filler to a receiving part adjacent to the channel 67, and then by hardening the filler.

When energy is provided to the valve 68, e.g., by emitting a laser, the exothermic minute particles generate heat rapidly, and then the filler is rapidly liquidized by the heat. This liquidized filler flows into the channel 67 and hardens there, thereby closing the channel 67 and preventing fluid from flowing through it. The microfluidic system 50 is provided with an external energy source 54 for providing energy to the valve 68. The energy source 54 may be configured to emit electromagnetic waves to the valve 68. Specifically, the energy source 54 may include a laser source such as a laser diode to emit a laser to the valve 68.

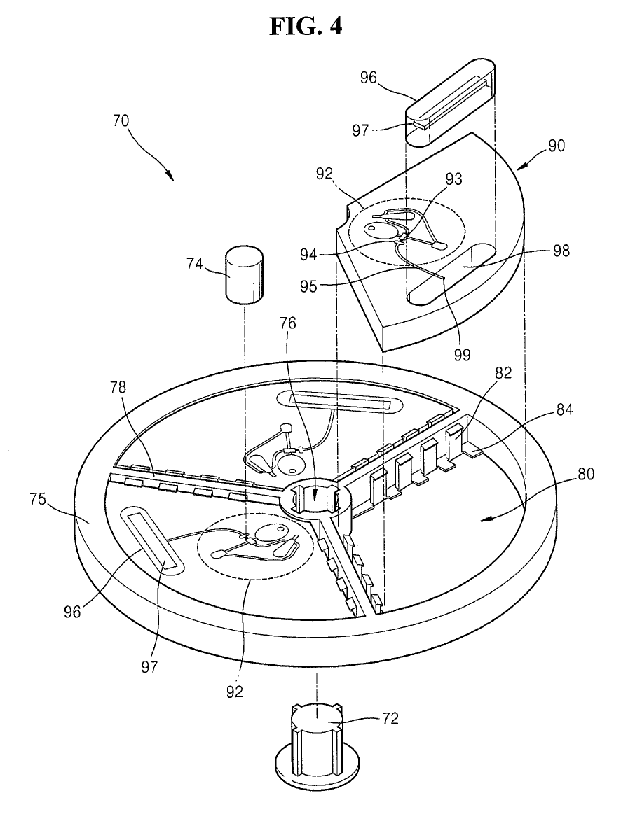

FIG. 4 is an exploded perspective view of a microfluidic system according to another exemplary embodiment.

As shown in FIG. 4, a microfluidic system 70 according to another exemplary embodiment includes a spindle motor 72, a rotatable frame 75 detachably connected to the motor 72, and at least one bio cartridge 90 detachably mounted in the frame 75.

The frame 75 includes a mounting hole 76 accommodating the spindle motor 72 at the center of the frame, a plurality of partition walls 78 extending radially from that center, and a plurality of cells 80 separated by the walls 78 and having dimensions and fanwise shape. The cell 80 includes hook members 82 detachably securing the bio cartridge 90 in the cell, and a bracket 84 supporting the bio cartridge 90.

The bio cartridge 90 is mounted in at least one of the cells 80 of the frame 75. The bio cartridge 90 has a fanwise shape corresponding to the shape of the cell 80. The bracket 84 supports the bio cartridge 90 mounted in the cell 80, and the hook members 82 secure the bio cartridge 90 in the cell 80 and prevent the bio cartridge 90 from becoming unintentionally detached from the cell 80. The bio cartridge 90 may be separated from the frame 75 by deforming the hook members 82 outwards and lifting up the bio cartridge 90.

The bio cartridge 90 includes a chamber retaining a small quantity of fluid, a channel transporting the fluid, a valve controlling the flow of the fluid, and a test kit 96 detachably loaded on the bio cartridge 90. Specifically, the bio cartridge 90 depicted in FIG. 4 includes a separating unit 92 centrifugally separating a sample such as whole blood (WB), a groove 98 accommodating the test kit 96, and a channel 95 connecting the separating unit 92 to the groove 98.

The test kit 96 has a test strip 97 therein, which reacts with a particular substance contained in the fluid that is extracted from the separating unit 92, and then determines the existence and the amount of the particular substance. The fluid extracted from the separating unit 92 flows, through the channel 95 and through an outlet 99 formed on the accommodating groove 98, into the test kit 96. If there is a particular substance desired for detection, the test strip 97 will react with that substance and change to be distinguishable.

The bio cartridge 90 also includes a valve 93 controlling the flow of the fluid through the channel 95.

The valve 93 opens the channel 95 under a certain condition. The valve includes a phase transition material, which remains in a solid phase at normal temperature, and a number of exothermic minute particles dispersed in the phase transition material. Since a valve filler for forming the valve 93 is the same as the filler for the valve 33 in FIG. 1, a description thereof will be omitted. The valve 93 may be formed by injecting the liquidized filler to the channel 95, and then hardening the filler.

When energy is provided to the valve 93, e.g., by emitting a laser, the exothermic minute particles generate heat rapidly, and then the filler is rapidly liquidized by the heat. The liquidized filler is discharged to a drain 94 provided on the channel 95, thereby opening the channel 95 so that the fluid flows. The microfluidic system 70 is provided with an external energy source 74 for applying energy to the valve 93. The energy source 74 may be configured to emit electromagnetic waves to the valve 93. Specifically, the source 74 may include a laser source such as a laser diode to emit a laser to the valve 93.

While the exemplary embodiments have been shown and described, it will be understood by those skilled in the art that various changes in form and details may be made thereto without departing from the spirit and scope of this disclosure as defined by the appended claims.

In addition, many modifications can be made to adapt a particular situation or material to the teachings of this disclosure without departing from the essential scope thereof. Therefore, it is intended that this disclosure not be limited to the particular exemplary embodiments disclosed as the best mode contemplated for carrying out this disclosure, but that this disclosure will include all embodiments falling within the scope of the appended claims.

What is claimed is:

1. A microfluidic system comprising:
a spindle motor;
a frame detachably connected to the spindle motor and comprising a plurality of cells separated by partition walls; and
at least one cartridge detachably accommodated in at least one of the plurality of cells,
wherein the cartridge comprises a chamber configured to store a fluid, a channel configured to transport the fluid, and a valve configured to control a flow of the fluid within the channel.

2. The microfluidic system according to claim 1, wherein the valve comprises a phase transition material, and a plurality of minute particles which are dispersed in the phase transition material and generate heat when energy is applied thereto.

3. The microfluidic system according to claim 2, wherein the minute particles are metal oxides.

4. The microfluidic system according to claim 2, wherein the phase transition material is wax, gel, or thermoplastic resin.

5. The microfluidic system according to claim 2, further comprising an external energy source which applies energy to the valve so that the minute particles absorb the energy and thereby generate heat which causes the phase transition material to undergo a phase transition to be able to flow.

6. The microfluidic system according to claim 5, wherein the energy source is configured to emit electromagnetic waves to the valve.

7. The microfluidic system according to claim 1, further comprising a dummy cartridge detachably accommodated in at least one of the cells which is not loaded with the cartridge, so as to control a rotational balance of the frame.

8. The microfluidic system according to claim 1, wherein the cell has a fanwise shape extending from a rotational center of the frame, and the cartridge has a fanwise shape corresponding to the shape of the cell.

9. The microfluidic system according to claim 1, further comprising a fixing portion which detachably secures the cartridge in the cell, the fixing portion comprising at least one hook member disposed inside of the cell.

10. The microfluidic system according to claim 1, wherein further comprising a cover member which is detachably coupled to the frame to close the cell thereby securing the cartridge in the cell.

11. The microfluidic system according to claim 1, further comprising a test kit detachably mounted in the cartridge and including a test strip for indicating an existence of a particular substance.

12. A bio cartridge detachably accommodated in a cell of a frame of a microfluidic device, the bio cartridge comprising:
a chamber configured to store a fluid;
a channel configured to transport the fluid; and
a valve configured to control a flow of the fluid within the channel.

13. The bio cartridge according to claim 12, wherein the valve comprises a phase transition material, and a plurality of minute particles which are dispersed in the phase transition material and generate heat when energy is applied thereto.

14. The bio cartridge according to claim 13, wherein the minute particles are minute metal oxides.

15. The bio cartridge according to claim 13, wherein the phase transition material is wax, gel, or thermoplastic resin.

16. The bio cartridge according to claim 13, wherein the bio cartridge has a fanwise shape corresponding to a shape of the cell of the frame.

17. The bio cartridge according to claim 13, further comprising a test kit detachably mounted in the bio cartridge and including a test strip for indicating an existence of a particular substance.

18. A microfluidic device comprising:
a frame having a disk shape, the frame comprising at least one cell; and
at least one cartridge detachably accommodated in the at least one cell, the cartridge comprising a test unit configured to perform a test on a sample fluid based on centrifugal force.

19. The microfluidic device according to claim 18, wherein the test unit comprises a chamber configured to store a fluid, a channel configured to transport the fluid, and a valve configured to control a flow of the fluid within the channel.

20. The microfluidic device according to claim 19, wherein the valve comprises a phase transition material, and a plurality of minute particles which are dispersed in the phase transition material and generate heat when energy is applied thereto.

21. The microfluidic device according to claim 18, wherein the frame comprises a plurality of cells separated by partition walls.

22. The microfluidic device according to claim 18, further comprising a fixing portion which detachably secures the cartridge in the cell, the fixing portion comprising at least one hook member disposed inside of the cell.

23. The microfluidic device according to claim 18, further comprising a cover member which is detachably coupled to the frame to close the cell thereby securing the cartridge in the cell.
